(19) United States
(12) Patent Application Publication
(10) Pub. No.: US 2017/0252287 A1
(43) Pub. Date: Sep. 7, 2017

(54) HAIR CARE REGIMEN USING CONDITIONER COMPRISING SILICONE RESIN AND AMINOSILICONE

(71) Applicant: The Procter & Gamble Company, Cincinnati, OH (US)

(72) Inventors: Jun Ji Zhang, Singapore (SG); Martin Chang Shou Lee, Singapore (SG); Lisa Jo Bartz, Singapore (SG); Christina Marie Gemmer, Lebanon, OH (US); Erica Vencil Buckner, Singapore (SG); Brian Xiaoqing Song, Mason, OH (US)

(21) Appl. No.: 15/448,925

(22) Filed: Mar. 3, 2017

Related U.S. Application Data
(60) Provisional application No. 62/303,401, filed on Mar. 4, 2016.

(51) Int. Cl. A61K 8/898; A61Q 5/12; A61K 8/41; A61K 8/73; A61K 8/44; A61K 8/46; A61Q 5/02; A61K 8/89

(52) U.S. Cl. CPC A61K 8/898; A61Q 5/02; A61Q 5/12; A61K 8/89; A61K 8/731; A61K 8/44; A61K 8/463; A61K 8/416; A61K 2800/30; A61K 2800/5426; A61K 2800/524; A61K 2800/594; A61K 2800/596; A61K 2800/884

(57) ABSTRACT

Disclosed is a method of treating the hair, the method comprising: a) applying to the hair a shampoo composition comprising a detersive surfactant, wherein the shampoo composition is substantially free of silicone conditioning agents and/or cationic conditioning polymers; b) rinsing the shampoo composition from the hair; c) applying to the hair a conditioning composition comprising a silicone resin and an aminosilicone, wherein the aminosilicone has non-quaternized and/or quaternary amine functional groups. The present invention provides improved conditioning benefits such as hair manageability and/or reduced frizziness/friction.

### FIELD OF THE INVENTION

Described herein is a method of treating the hair, the method comprising: a) applying to the hair a shampoo composition comprising a detersive surfactant, wherein the shampoo composition is substantially free of silicone conditioning agents and/or cationic conditioning polymers; b) rinsing the shampoo composition from the hair; c) applying to the hair a conditioning composition comprising a silicone resin and an aminosilicone, wherein the aminosilicone has non-quaternized and/or quaternary amine functional groups. The present invention provides improved conditioning benefits such as hair manageability and/or reduced frizziness/friction.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits.

For example, WO2009/016555 discloses a conditioning composition comprising: (a) a surfactant system comprising: di- and mono-alkyl quaternized ammonium salt cationic surfactants; (b) a high melting point fatty compound; (c) an aminosilicone; (d) a silicone resin; and (e) an aqueous carrier, and the composition is said to provide improved wet and dry conditioning benefits while providing durable conditioning and chronic/long lasting color protection benefit.

Such conditioning agents are often provided to a hair through conditioning compositions applied after shampooing the hair. However, there remains a need to effectively deliver conditioning benefits to the hair from the conditioning compositions.

### SUMMARY OF THE INVENTION

The present invention is directed to a method of treating the hair, the method comprising the following steps: a) applying to the hair a shampoo composition comprising a detersive surfactant, wherein the shampoo composition is substantially free of silicone conditioning agents and/or cationic conditioning polymers; b) rinsing the shampoo composition from the hair; c) applying to the hair a conditioning composition comprising a silicone resin and an aminosilicone, wherein the aminosilicone has non-quaternized and/or quaternary amine functional groups.

The present invention provides improved conditioning benefits such as hair manageability and/or reduced frizziness/friction. The inventors of the present invention surprisingly found a method to effectively deliver conditioning benefits to the hair, especially the conditioning benefits from a specific silicone combination.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

### Method of Treating Hair

The method of treating the hair described herein comprises the following steps: a) applying to the hair the specific shampoo composition; b) rinsing the specific shampoo composition from the hair; and c) then applying to the hair the specific conditioning composition.

Preferably, in the method of the present invention, no composition is applied between the step b) “rinsing the specific shampoo composition from the hair” and the step c) “then applying to the hair the specific conditioning composition”.

The specific conditioning composition can be a leave-on or rinse-off product, preferably rinse-off product. When the specific conditioning composition is a rinse-off product, preferably, the step c) further comprises: rinsing the specific conditioning composition from the hair.

The method of the present invention can further comprise a step d) applying to the hair an additional conditioning composition, after applying the above specific conditioning composition. This additional conditioning composition can be leave-on or rinse-off, preferably rinse-off. When the additional conditioning composition is a rinse-off product, the step d) further comprises: rinsing the additional conditioning composition from the hair. This additional conditioning can be applied to the hair before rinsing the specific conditioning composition from the hair, and then rinsed off from the hair together with the specific conditioning composition. Alternatively, the additional conditioning composition can be applied after rinsing the specific conditioning composition from the hair, then, rinsing from the hair when the additional conditioning composition is a rinse-off product.

The method of the present invention can further comprise a step e) applying a leave-on conditioning composition to the hair. This leave-on conditioning composition can be applied with or without the additional conditioning composition described above in the step d).

The details of shampoo and conditioning compositions are described below.

### Shampoo Compositions

Shampoo compositions useful herein comprise a detersive surfactant, and are substantially free of silicone conditioning agents or substantially free of cationic conditioning polymers. Preferably, the shampoo compositions useful herein are substantially free of silicone conditioning agent and cationic conditioning polymers.

### Detersive Surfactants

The shampoo composition comprises a detersive surfactant. The detersive surfactant can be included at a level of from about 8% to about 40%, preferably from about 10% to about 40%, more preferably from about 12% to about 36%, still more preferably from about 14% to about 32% by weight of the shampoo composition.

Suitable detersive surfactants herein include, for example, anionic surfactants, amphoteric surfactants, zwitterionic surfactants, nonionic surfactant, and mixtures thereof, preferably, anionic surfactants, amphoteric surfactants, zwitterionic surfactants, and mixtures thereof.

Anionic surfactants useful herein include, but are not limited to, undecyl sulfate compound selected from the group consisting of:

a) R₁ O(CH₂CHR₃O)_{y} SO₃M;

b) CH₃ (CH₂)_{z} CHR₂ CH₂ O (CH₂ CHR₃O)_{y} SO₃M; and

c) mixtures thereof,

where R₁ represents CH₃ (CH₂)₁₀, R₂ represents H or a hydrocarbon radical comprising 1 to 4 carbon atoms such that the sum of the carbon atoms in z and R₂ is 8, R₃ is H or CH₃, y is 0 to 7, the average value of y is about 1 when y is not zero (0), and M is a monovalent or divalent, positively-charged cation.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the hair care composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment, the anionic surfactant is sodium lauryl sulfate or sodium laureth sulfate.

The detersive surfactants preferably comprise anionic surfactants, and more preferably further comprises amphoteric and/or zwitterionic surfactants as co-surfactants. Such co-surfactants, if included, can be included at a level of from about 0.25% to about 15%, preferably from about 0.5% to about 15%, more preferably from about 1% to about 12%, still more preferably from about 2% to about 10% by weight of the shampoo composition.

Amphoteric surfactants suitable herein include those surfactants described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Suitable amphoteric surfactant include, but are not limited to, those selected from the group consisting of: sodium cocaminopropionate, sodium cocaminodipropionate, sodium cocoamphoacetate, sodium cocoamphohydroxypropylsulfonate, sodium cocoamphopropionate, sodium comamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium comamphopropionate, ammonium lauraminopropionate, ammonium lauroamphoacetate, ammonium lauroamphohydmxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine comamphopropionate, triethanonlamine lauraminopropionate, triethanonlamine lauroamphoacetate, triethanonlamine lauroamphohydroxypropylsulfonate, triethanonlamine lauroamphopropionate, triethanonlamine comamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, lauryl diethylenediaminoglycine, and mixtures thereof

The amphoteric co-surfactant can be a surfactant according to the following structure:

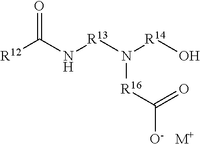

wherein R12 is a C-linked monovalent substituent selected from the group consisting of substituted alkyl systems comprising 9 to 15 carbon atoms, unsubstituted alkyl systems comprising 9 to 15 carbon atoms, straight alkyl systems comprising 9 to 15 carbon atoms, branched alkyl systems comprising 9 to 15 carbon atoms, and unsaturated alkyl systems comprising 9 to 15 carbon atoms; R13, R14, and R15 are each independently selected from the group consisting of C-linked divalent straight alkyl systems comprising 1 to 3 carbon atoms, and C-linked divalent branched alkyl systems comprising 1 to 3 carbon atoms; and M+ is a monovalent counterion selected from the group consisting of sodium, ammonium and protonated triethanolamine. In an embodiment, the amphoteric surfactant is selected from the group consisting of: sodium cocoamphoacetate, sodium cocoamphodiacetate, sodium lauroamphoacetate, sodium lauroamphodiacetate, ammonium lauroamphoacetate, ammonium cocoamphoacetate, triethanolamine lauroamphoacetate, triethanolamine cocoamphoacetate, and mixtures thereof.

Zwitterionic surfactant suitable herein is a derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. The zwitterionic surfactant can be selected from the group consisting of: cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and mixtures thereof. A suitable zwitterionic surfactant is lauryl hydroxysultaine. The zwitterionic surfactant can be selected from the group consisting of: lauryl hydroxysultaine, cocamidopropyl hydroxysultaine, coco-betaine, coco-hydroxysultaine, coca-sultaine, lauryl betaine, lauryl sultaine, and mixtures thereof.

Other than the above exemplified as detersive surfactant and/or co-surfactant, non-ionic surfactant may be used as a co-surfactant of anionic surfactants. Such nonionic co-surfactant can be selected from the group consisting of: Cocamide, Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Oleamide, PPG-2 Cocamide, PPG-2 Hydroxyethyl Cocamide, and mixtures thereof.

Shampoo being Substantially Free of Silicone Conditioning Agents and/or Cationic Conditioning Polymers

The shampoo compositions useful herein are substantially free of silicone conditioning agents or substantially free of cationic conditioning polymers. Preferably, the shampoo compositions useful herein are substantially free of silicone conditioning agent and cationic conditioning polymers.

In the present invention, “the shampoo composition being substantially free of silicone conditioning agents” means that: the shampoo composition is free of silicone conditioning agents; or, if the shampoo composition contains silicone conditioning agents, the level of such silicone conditioning agents is very low. In the present invention, a total level of such silicone conditioning agents, if included in the shampoo compositions, preferably 0.1% or less, more preferably 0.05% or less, still more preferably 0.01% or less, even more preferably 0.005% or less by weight of the shampoo composition. Most preferably, the total level of such silicone conditioning agents is 0% by weight of the shampoo composition.

In the present invention, “the shampoo composition being substantially free of cationic conditioning polymers” means that: the shampoo composition is free of cationic conditioning polymers; or, if the shampoo composition contains cationic conditioning polymers, the level of such cationic conditioning polymers is very low. In the present invention, a total level of such cationic conditioning polymers, if included in the shampoo compositions, preferably 0.01% or less, more preferably 0.005% or less by weight of the shampoo composition. Most preferably, the total level of such cationic conditioning polymers is 0% by weight of the shampoo composition.

Such silicone conditioning agents are, for example, polyalkyl siloxanes, polyaryl siloxanes, polyalkylaryl siloxanes, polyether siloxane copolymers, aminosilicones, aminosilicone copolyols, quaternized aminosilicones, quaternized aminosilicone copolyols, and mixtures thereof.

Such cationic conditioning polymers are, for example: cationic guar polymers, cationic non-guar galactomannan polymers, cationic tapioca polymers, cationic copolymers of acrylamide monomers and cationic monomers, cationic cellulose polymers, and combinations thereof.

### High Melting Point Fatty Compounds in Shampoo

The shampoo composition may contain high melting point fatty compounds, however, the level of such high melting point fatty compounds can be limited up to about 3%, preferably up to about 2%, more preferably up to about 1%, still more preferably up to about 0.5%, even more preferably up to about 0.25% by weight of the shampoo composition. The shampoo composition may be preferably substantially free of shampoo high melting point fatty compounds, and more preferably may comprise 0% shampoo high melting point fatty compounds, by weight of the shampoo composition. Such high melting point fatty compounds are explained in detail later for conditioning compositions.

### Conditioning Compositions

The conditioning compositions used in the step b) useful herein comprise silicone resins and aminosilicones. Preferably, the conditioning compositions useful herein further comprises cationic surfactants and high melting point fatty compounds.

Preferably, when the conditioning compositions contain cationic surfactants and/or gel matrix formed by cationic surfactants and high melting point fatty compounds, the composition of the present invention is substantially free of anionic surfactants, in view of avoiding undesirable interaction with cationic surfactants and/or in view of stability of the gel matrix. In the present invention, “the conditioning composition being substantially free of anionic surfactants” means that: the conditioning composition is free of anionic surfactants; or, if the conditioning composition contains anionic surfactants, the level of such anionic surfactants is very low. In the present invention, a total level of such anionic surfactants, if included in the conditioning compositions, preferably 1% or less, more preferably 0.5% or less, still more preferably 0.1% or less by weight of the conditioning composition. Most preferably, the total level of such anionic surfactants is 0% by weight of the conditioning composition.

### Silicone Resin

The conditioning compositions of the present invention comprise a silicone resin. Without wishing to be bound by theory, silicone resins are believed to create a 3-dimensional network within the aminosilicone fluid giving rise to viscoelasticity thereby improving the adhesive properties of the fluid and hence the conditioning benefits on a fibrous substrate. Preferably, the silicone resin is insoluble in water. In the case that the fiber treatment composition is an emulsion, the mixture of the aminosilicone and the silicone resin may be dispersed there in the form of emulsified droplets.

Preferably, the organosiloxane resins according to the invention are solid at about 25° C. Whilst not wishing to be bound to theory, it is believed that solid silicone resin can form an ultrafine dispersion in the aminosilicone which behaves unlike any of the silicone resin per se, the aminosilicone per se, and a blended fluid with the aminosilicone when using fluid silicone resin.

Preferably, the organosiloxane resins according to the invention have a molecular weight range of from about 500 to about 50,000, more preferably from about 750 to about 25,000, still more preferably from about 1,000 to about 10,000 grams/mole. Whilst not wishing to be bound to theory, it is believed that silicone resins of lower or larger molecular weight tend to provide reduced synergy with the aminosilicone.

The silicone resin is included in the composition at levels by weight of the composition of from about 0.0001% to about 10%, preferably from about 0.001% to about 5%, more preferably from about 0.002% to about 3%, still more preferably from about 0.003% to about 1%. It is preferred to contain the silicone resin such that the weight ratio of the silicone resin to the aminosilicone is within the scope of from about 1:500 to about 1:3000, more preferably from about 1:800 to about 1:2000, still more preferably from about 1:800 to about 1:1500. It is believed that a lower level of the silicone resin provides reduced synergetic benefit with the aminosilicone, while a higher level of the silicone resin tend to provide poor sensory feel.

Organosiloxane resins useful herein are combinations of R₃SiO_{1/2} “M” units, R₂SiO “D” units, RsiO_{3/2} “T” units, SiO₂ “Q” units in ratios to each other that satisfy the relationship RₙSiO_{(4-n)/2} where n is a value between 1.0 and 1.50 and R is a methyl group. Silanol or alkoxy functionalities may also be present in the resin structure.

More preferably, the organosiloxane resins comprise repeating monofunctional R₃SiO_{1/2} “M” units and the quadrafunctional SiO₂ “Q” units, otherwise known as “MQ” resins. In this case, the ratio of the “M” to “Q” functional units is advantageously from 0.7 and the value of n is 1.2. Organosiloxane resins such as these are commercially available as SR1000 available from GE Bayer Silicones and Wacker 803 from Wacker Silicones.

### Aminosilicone

The conditioning compositions of the present invention comprise an aminosilicone. The aminosilicone is included in the composition at levels by weight of the composition of from about 0.1% to about 20%, preferably from about 0.25% to about 15%, more preferably from about 0.5% to about 10%, still more preferably from about 1% to about 7%. The aminosilicones useful herein have non-quaternized amine functional groups such as primary, secondary, tertiary amine functional groups, and/or quaternary amine functional groups. Preferably, the aminosilicones useful herein are free of quaternary amine functional groups, thus, have non-quaternized amine functional groups such as primary, secondary, and/or tertiary amine functional groups.

Preferably, the aminosilicones useful have the amine functional groups: (i) as pendant groups attaching to a polysiloxane backbone; or (ii) at terminal ends of a polysiloxane backbone. More preferred herein are (i) aminosilicones having aminofunctional groups as pendant group attaching to a polysiloxane backbone.

The aminosilicone useful herein may be preferably free of copolyol groups such as polyethylene glycol groups and polypropylene glycol groups, i.e., contain 0% of copolyol groups.

Such aminosilicones having non-quaternized aminofunctional groups as pendant group attaching to a polysiloxane backbone useful herein include, but are not limited to silicones of the following structure:

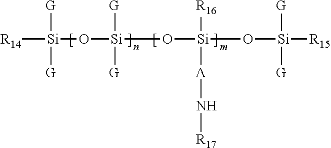

wherein:
a sum (n+m) ranges from about 2 to about 2000, preferably from about 150 to about 2000, more preferably from about 250 to about 1200, still more preferably from about 300 to about 800;
n is a number ranging from about 1 to about 1999, and m is a number ranging from about 1 to about 1999; and n and m are chosen such that a ratio of m:n is from about 1:1000 to about 1:10, preferably from about 1:1000 to about 1:25, more preferably from about 1:800 to about 1:50, still more preferably from about 1:500 to about 1:50, even more preferably from about 1:400 to about 1:100;
R₁₄, R₁₅, R₁₆, which may be identical or different, are chosen from a hydroxyl radical, C1-C4 alkoxy radicals and methyl, preferably R₁₄ and R₁₅ are hydroxyl radical and/or C1-C4 alkoxy radicals and R₁₆ is methyl;
A is chosen from linear and branched C3-C8 alkenyl radicals;

R₁₇ is chosen from H, phenyl, linear or branched C1-C4 alkyl radical, benzyl or preferably linear or branched (C2-C8)NH₂; and

G is chosen from H, phenyl, hydroxyl, C1-C8 alkyl, preferably methyl.
These aminosilicones may be of the random or block type.

Suitable aminosilicones of the present invention include, but are not limited to, organomodified silicones with amine functionality available commercially under the trade names such as ADM1100 and ADM1600 from Wacker Silicones, AP6087, DC2-8211, DC8822, DC8822A, DC8803, DC2-8040, DC2-8813, DC2-8630 and DC8566 from Dow Corning Corporation, KF-862, KF-861, KF-8625, KF-8005, KF-8004, KF-8675, KF-873, and X-52-2328 from Shin-Etsu Corporation, and TSF 4702, TSF 4703, TSF 4704, TSF 4705, TSF 4707, TSF 4708, TSF 4709, F42-B3115, SF 1708, SF 1923, SF 1921, SF 1925, OF TP AC3309, OF 7747, OF-NH TP AI3631, OF-NH TP AI3683 from GE Bayer Silicones.

Highly preferred aminosilicones of the present invention are organomodified silicones with amine functionality with viscosities of greater than about 4,000 mPa·s in view of conditioning efficiency and up to about 100,000 mPa·s in view of friendly incorporation processing and spreadability, which include, but are not limited to, commercially available fluids under the trade names ADM1100 from Wacker Silicones, AP6087, DC8803 from Dow Corning Corporation, and TSF 4707 from GE Bayer Silicones.

Aminosilicones having non-quaternized or quaternary amine functional groups at terminal ends of a polysiloxane backbone useful herein include, for example, those which conform to the general formula (I):

(R₁)ₐG₃₋ₐ-Si-(—OSiG₂)ₙ-(—OSiG_{b}(R₁)_{2-b})ₘ—O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Highly preferred amino silicones are those corresponding to formula (I) wherein m=0, a=1, q=3, G=methyl, n is preferably from about 1500 to about 1700, more preferably about 1600; and L is —N(CH₃)₂ or —NH₂, more preferably —NH₂. Another highly preferred amino silicones are those corresponding to formula (I) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones, as one or both ends of the silicone chain are terminated by nitrogen containing group.

The above aminosilicones have a viscosity of preferably from about 1,000 mPa·s to about 100,000 mPa·s, more preferably from about 5,000 mPa·s to about 50,000 mPa·s.

### Additional Silicone Compound

The compositions of the present invention may further contain other silicone compounds than the aminosilicones described above. The additional silicone compounds herein can be used at levels by weight of the composition of preferably from about 0.1% to about 20%, more preferably from about 0.5% to about 10%, still more preferably from about 1% to about 8%.

Such additional silicone compounds are, for example, polyalkyl siloxanes, polyaryl siloxanes, polyalkylaryl siloxanes, polyether siloxane copolymers, and mixtures thereof.

The additional silicone compounds useful herein, as a single compound, as a blend or mixture of at least two silicone compounds, or as a blend or mixture of at least one silicone compound and at least one solvent, have a viscosity of preferably from about 1 to about 2,000,000 mPa·s at 25° C.

The additional silicone compounds may further be incorporated in the present composition in the form of an emulsion.

### Cationic Surfactant

Preferably, the conditioning compositions of the present invention comprise a cationic surfactant. The cationic surfactant can be included in the composition at a level of from about 0.1%, preferably from about 0.5%, more preferably from about 0.8%, still more preferably from about 1.0%, and to about 20%, preferably to about 10%, more preferably to about 8.0%, still more preferably to about 6.0% by weight of the composition, in view of providing the benefits of the present invention.

Preferably, in the present invention, the surfactant is water-insoluble. In the present invention, “water-insoluble surfactants” means that the surfactants have a solubility in water at 25° C. of preferably below 0.5 g/100 g (excluding 0.5 g/100 g) water, more preferably 0.3 g/100 g water or less.

Cationic surfactant useful herein can be one cationic surfactant or a mixture of two or more cationic surfactants. Preferably, the cationic surfactant is selected from: a mono-long alkyl quaternized ammonium salt; a combination of a mono-long alkyl quaternized ammonium salt and a di-long alkyl quaternized ammonium salt; a mono-long alkyl amine; a combination of a mono-long alkyl amine and a di-long alkyl quaternized ammonium salt; and a combination of a mono-long alkyl amine and a mono-long alkyl quaternized ammonium salt.

### Mono-Long Alkyl Amine

Mono-long alkyl amine useful herein are those having one long alkyl chain of preferably from 12 to 30 carbon atoms, more preferably from 16 to 24 carbon atoms, still more preferably from 18 to 22 alkyl group. Mono-long alkyl amines useful herein also include mono-long alkyl amidoamines. Primary, secondary, and tertiary fatty amines are useful.

Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al.

These amines are used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid, at a molar ratio of the amine to the acid of from about 1:0.3 to about 1:2, more preferably from about 1:0.4 to about 1:1.

### Mono-Long Alkyl Quaternized Ammonium Salt

The mono-long alkyl quaternized ammonium salts useful herein are those having one long alkyl chain which has from 12 to 30 carbon atoms, preferably from 16 to 24 carbon atoms, more preferably C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

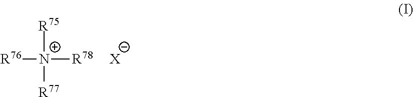

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Di-Long Alkyl Quaternized Ammonium Salts

When used, di-long alkyl quaternized ammonium salts are preferably combined with a mono-long alkyl quaternized ammonium salt and/or mono-long alkyl amine salt, at the weight ratio of from 1:1 to 1:5, more preferably from 1:1.2 to 1:5, still more preferably from 1:1.5 to 1:4, in view of stability in rheology and conditioning benefits.

Di-long alkyl quaternized ammonium salts useful herein are those having two long alkyl chains of from 12 to 30 carbon atoms, more preferably from 16 to 24 carbon atoms, still more preferably from 18 to 22 carbon atoms. Such di-long alkyl quaternized ammonium salts useful herein are those having the formula (I):

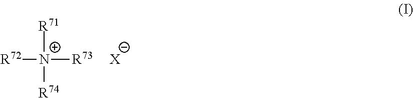

wherein two of R⁷¹, R⁷², R⁷³ and R⁷⁴ are selected from an aliphatic group of from 12 to 30 carbon atoms, preferably from 16 to 24 carbon atoms, more preferably from 18 to 22 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms, preferably from 1 to 3 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C1-C4 alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Preferably, two of R⁷¹, R⁷², R⁷³ and R⁷⁴ are selected from an alkyl group of from 12 to 30 carbon atoms, preferably from 16 to 24 carbon atoms, more preferably from 18 to 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Such preferred di-long alkyl cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### High Melting Point Fatty Compound

Preferably, the conditioning composition of the present invention comprises a high melting point fatty compound. The high melting point fatty compound can be included in the composition at a level of from about 1.0%, preferably from about 1.5%, more preferably from about 2%, still more preferably from about 3%, and to about 30%, preferably to about 15%, more preferably to about 8% by weight of the composition, in view of providing the benefits of the present invention.

The high melting point fatty compound useful herein have a melting point of 25° C. or higher, preferably 40° C. or higher, more preferably 45° C. or higher, still more preferably 50° C. or higher, in view of stability of the emulsion especially the gel matrix. Preferably, such melting point is up to about 90° C., more preferably up to about 80° C., still more preferably up to about 70° C., even more preferably up to about 65° C., in view of easier manufacturing and easier emulsification. In the present invention, the high melting point fatty compound can be used as a single compound or as a blend or mixture of at least two high melting point fatty compounds. When used as such blend or mixture, the above melting point means the melting point of the blend or mixture.

The high melting point fatty compound useful herein is selected from the group consisting of fatty alcohols, fatty acids, and mixtures thereof. Further, it is understood by the artisan that, depending on the number and position of double bonds, and length and position of the branches, certain compounds having certain required carbon atoms may have a melting point of less than the above preferred in the present invention. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols.

Preferred fatty alcohols include, for example, cetyl alcohol (having a melting point of about 56° C.), stearyl alcohol (having a melting point of about 58-59° C.), behenyl alcohol (having a melting point of about 71° C.), and mixtures thereof. These compounds are known to have the above melting point. However, they often have lower melting points when supplied, since such supplied products are often mixtures of fatty alcohols having alkyl chain length distribution in which the main alkyl chain is cetyl, stearyl or behenyl group.

In the present invention, more preferred fatty alcohol is a mixture of cetyl alcohol and stearyl alcohol.

Generally, in the mixture, the weight ratio of cetyl alcohol to stearyl alcohol is preferably from about 1:9 to 9:1, more preferably from about 1:4 to about 4:1, still more preferably from about 1:2.3 to about 1.5:1.

When using higher level of total cationic surfactant and high melting point fatty compounds, the mixture has the weight ratio of cetyl alcohol to stearyl alcohol of preferably from about 1:1 to about 4:1, more preferably from about 1:1 to about 2:1, still more preferably from about 1.2:1 to about 2:1, in view of avoiding to get too thick for spreadability. It may also provide more conditioning on damaged part of the hair.

### Aqueous Carrier

Preferably, the conditioning composition of the present invention comprises an aqueous carrier. The level and species of the carrier are selected according to the compatibility with other components, and other desired characteristic of the product.

The carrier useful in the present invention includes water and water solutions of lower alkyl alcohols. The lower alkyl alcohols useful herein are monohydric alcohols having 1 to 6 carbons, more preferably ethanol and isopropanol.

Preferably, the aqueous carrier is substantially water. Deionized water is preferably used. Water from natural sources including mineral cations can also be used, depending on the desired characteristic of the product. Generally, the compositions of the present invention comprise from about 40% to about 99%, preferably from about 50% to about 95%, and more preferably from about 70% to about 90%, and more preferably from about 80% to about 90% water.

### Gel Matrix

Preferably, in the conditioning composition of the present invention, a gel matrix is formed by the cationic surfactant, the high melting point fatty compound, and an aqueous carrier. The gel matrix is suitable for providing various conditioning benefits, such as slippery feel during the application to wet hair and softness and moisturized feel on dry hair.

Preferably, when the gel matrix is formed, the cationic surfactant and the high melting point fatty compound are contained at a level such that the weight ratio of the cationic surfactant to the high melting point fatty compound is in the range of, preferably from about 1:1 to about 1:10, more preferably from about 1:1.5 to about 1:7, still more preferably from about 1:2 to about 1:6, in view of providing improved wet conditioning benefits.

### Additional Components

The compositions of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; coloring agents, such as any of the FD&C or D&C dyes; perfumes; ultraviolet and infrared screening and absorbing agents such as benzophenones; and antidandruff agents such as zinc pyrithione; non-ionic surfactant such as mono-9-octadecanoate poly(oxy-1,2-ethanediyl) supplied as, for example, Tween 20; and buffer such as aminomethyl propanol.

### Additional Conditioning Composition in Step D

The additional conditioning compositions used in step d) can be anything, preferably comprises silicone compounds such as aminosilicones described above and/or non-amino silicones described above as “ADDITIONAL SILICONE COMPOUNDS”, more preferably comprises aminosilicones described above.

### Leave-on Conditioning Composition in Step E

The leave-on conditioning compositions used in step e) can be anything, preferably comprises silicone compounds such as aminosilicones described above and/or non-amino silicones described above as “ADDITIONAL SILICONE COMPOUNDS”, more preferably comprises aminosilicones described above.

### Product Forms

The shampoo and conditioning compositions of the present invention can be formulated in a wide variety of product forms, including but not limited to liquids, creams, gels, emulsions, mousses and sprays.

The conditioner compositions of the present invention can be used as leave-on or rinse-off, preferably rinse-off.

### Key Features of the Invention

A. The present invention is directed to a method of treating the hair, the method comprising the following steps:
   a) applying to the hair a shampoo composition comprising a detersive surfactant, wherein the shampoo composition is substantially free of silicone conditioning agents and/or cationic conditioning polymers, preferably wherein the shampoo composition is substantially free of silicone conditioning agents and cationic conditioning polymers;
   b) rinsing the shampoo composition from the hair;
   c) applying to the hair a conditioning composition comprising a silicone resin and an aminosilicone, wherein the aminosilicone has non-quaternized and/or quaternary amine functional groups, and preferably rinsing the conditioning composition from the hair.
B. The method of the preceding feature, further comprising a step:
   d) applying an additional conditioning composition to the hair, and rinsing the additional conditioning composition from the hair;
C. The method of the preceding feature, further comprising a step:
   e) applying an additional leave-on conditioning composition to the hair.
D. The method of any of the preceding features, wherein no composition is applied between the step (b) “rinsing the shampoo composition from the hair” and the step (c) “applying to the hair the conditioning composition”.
E. The method of any of the preceding features, wherein the aminosilicone has non-quaternized amine functional groups.
F. The method of any of the preceding features, wherein the aminosilicone has the amine functional groups: (i) as pendant groups attaching to a polysiloxane backbone; or (ii) at terminal ends of a polysiloxane backbone
G. The method of any of the preceding features, wherein the conditioning composition further comprises: a cationic surfactant; a high melting point fatty compound; and an aqueous carrier.

### Examples

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

Shampoo compositions (wt %)

| | SH | SH | SH | SH | SH |
|---|---|---|---|---|---|
| | Ex. 1 | Ex. 2 | Ex. 3 | CEx. i | CEx. ii |
| Water | q.s. | q.s. | q.s. | q.s. | q.s. |
| Sodium Laureth-3 Sulfate | 6 | 6 | 6 | 6 | — |
| (SLE3S) | | | | | |
| Sodium Lauryl Sulfate (SLS) | 6.5 | 6.5 | 6.5 | 6.5 | — |
| Ammonium Laureth-3 Sulfate | — | — | — | — | 8.5 |
| (ALE3S) | | | | | |
| Ammonium Lauryl Sulfate | — | — | — | — | 1.5 |
| (ALES) | | | | | |
| Cocoamidopropyl Betaine | 1 | 1 | 1 | 1 | 1.5 |
| Sodium Lauryl | — | — | — | — | 1.5 |
| Amphoacetate | | | | | |
| Cocamide MEA | 0.85 | 0.85 | 0.85 | 0.85 | — |
| Silicone microemulsion *1 | — | 2 | — | 2 | 0.5 |
| Cationic polymer *2 | — | — | 0.5 | 0.5 | 0.4 |
| Sodium citrate | 0.4 | 0.4 | 0.4 | 0.4 | 0.4 |
| Sodium Chloride | 1.0 | 1.0 | 1.0 | 1.0 | 1.0 |
| Glycol Distearate | — | — | — | — | 1.5 |
| Fragrance | 0.7 | 0.7 | 0.7 | 0.7 | 0.7 |
| Preservatives, pH adjusters | Up to | Up to | Up to | Up to | Up to |
| | 1% | 1% | 1% | 1% | 1% |

Conditioning compositions (wt %)

| | CN | CN | CN | CN |
|---|---|---|---|---|
| Components | Ex. 1 | Ex. 2 | Ex. 3 | CEx. i |
| MQ resin * 3 | 0.007 | 0.007 | 0.1 | 0 |
| Aminosilicone-1 * 4 | 7 | — | 7 | 7 |
| Aminosilicone-2 * 5 | — | 7 | — | — |
| Stearamidopropyldimethylamine | 2.0 | 2.0 | — | 2.0 |
| Behenyl trimethylammonium | — | — | 2.97 | — |
| methosulfate | | | | |
| Dicetyl dimethyl ammonium chloride | — | — | 0.75 | — |
| Glutamic Acid | 0.6 | 0.6 | — | 0.6 |
| Cetyl alcohol | 2.5 | 2.5 | 1.17 | 2.5 |
| Stearyl alcohol | 4.5 | 4.5 | 2.94 | 4.5 |
| Benzyl alcohol | 0.4 | 0.4 | 0.4 | 0.4 |
| Preservative | 0.03 | 0.03 | 0.03 | 0.03 |
| Perfume | 0.8 | 0.8 | 0.8 | 0.8 |
| Panthenol | — | — | 0.5 | — |

| | |
|---|---|
| Deionized Water | q.s. to 100% of the composition |

### Definitions of Components

*1 Silicone microemulsion: DC1664 available from Dow Corning
*2 Cationic polymer: Polyquaternium-10 having MW of 2,000,000 and CD of 2.0 meq/g
*3 MQ resin: SR1000 (Polytrimethyl hydrosilylsilicate) from GE Silicones
*4 Aminosilicone-1: ADM1100 from Wacker Silicones
*5 Aminosilicone-2: Available from GE having a viscosity 10,000 mPa·s, and having following formula (I):

(R₁)ₐG₃₋ₐ-Si-(—OSiG₂)ₙ-(—OSiG_{b}(R₁)_{2-b})ₘ—O—SiG₃₋ₐ(R₁)ₐ (I)

wherein G is methyl; a is an integer of 1; b is 0, 1 or 2, preferably 1; n is a number from 400 to about 600; m is an integer of 0; R₁ is a monovalent radical conforming to the general formula CqH_{2q}L, wherein q is an integer of 3 and L is —NH₂

### Method of Preparation

The exemplified shampoo and conditioning compositions can be prepared by conventional formulation and mixing techniques.

### Properties and Conditioning Benefits

For some of the above compositions, properties and benefits are evaluated by the following methods. Results of the evaluation are also shown below.

The method of the present invention shows improved conditioning such as reduced frizziness and/or improved hair manageability compared to comparative method.

### Reduced Frizziness on Dry Hair

Reduced frizziness on dry hair is evaluated by a panelist test. 8 panelists evaluated samples prepared by applying 0.1 ml of the above compositions per 1 g of wet hair. Panelists evaluated each sample from 1 (very poor) to 5 (very good, reduced frizziness) by touch. The data from the panelists were gathered, averaged, and scored, and compared.

### Hair Manageability

Hair manageability is evaluated by a visual inspection by panelists of the samples as follows:

Better hair manageability means less fly-away hairs and/or irregular hairs spreading out from main bulk of the hair.

| | Step (a) | Step (b) | Step (c) | | | |
|---|---|---|---|---|---|---|
| | Applying | Rinsing the | Applying | Step (d) | | |
| | below | shampoo | below | Rinsing the | | |
| | shampoo | composition | conditioning | conditioning | | |
| | composition | from the | composition | composition | Reduced | Hair |
| | to hair | hair | to the hair | from the hair | frizziness | Manageability |
| Method | SH Ex. 1 | See left | CN Ex. 1 | See left | Reduced | All panelists |
| Ex. 1 | | | | | frizziness | answered that |
| | | | | | scored | Method Ex. 1 |
| | | | | | 3.7 | shows better |
| | | | | | | hair |
| | | | | | | manageability |
| | | | | | | than Method |
| | | | | | | Ex. 8 |
| Method | SH Ex. 2 | See left | CN Ex. 1 | See left | Reduced | — |
| Ex. 2 | | | | | frizziness | |
| | | | | | scored 2.9 | |
| Method | SH Ex. 3 | See left | CN Ex. 1 | See left | — | — |
| Ex. 3 | | | | | | |
| Method | SH CEx. i | See left | CN Ex. 1 | See left | Reduced | — |
| Ex. 4 | | | | | frizziness | |
| | | | | | scored 2.4 | |
| Method | SH CEx. ii | See left | CN Ex. 1 | See left | Reduced | — |
| Ex. 5 | | | | | frizziness | |
| | | | | | scored 1.9 | |
| Method | SH Ex. 1 | See left | CN Ex. 2 | See left | — | — |
| Ex. 6 | | | | | | |
| Method | SH Ex. 1 | See left | CN Ex. 3 | See left | — | — |
| Ex. 7 | | | | | | |
| Method | SH Ex. 1 | See left | CN CEx. i | See left | — | All |
| Ex. 8 | | | | | | panelists |
| | | | | | | answered that |
| | | | | | | Method Ex. 1 |
| | | | | | | shows better |
| | | | | | | hair |
| | | | | | | manageability |
| | | | | | | than |
| | | | | | | Method |
| | | | | | | Ex. 8 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating the hair, the method comprising the following steps:
a) applying to the hair a shampoo composition comprising a detersive surfactant, wherein the shampoo composition is substantially free of silicone conditioning agents;
b) rinsing the shampoo composition from the hair;
c) applying to the hair a conditioning composition comprising a silicone resin and an aminosilicone, wherein the aminosilicone has non-quaternized and/or quaternary amine functional groups.

2. A method of treating the hair, the method comprising the following steps:
a) applying to the hair a shampoo composition comprising a detersive surfactant, wherein the shampoo composition is substantially free of cationic conditioning polymers;
b) rinsing the shampoo composition from the hair;
c) applying to the hair a conditioning composition comprising a silicone resin and an aminosilicone, wherein the aminosilicone has non-quaternized and/or quaternary amine functional groups.

3. A method of treating the hair, the method comprising the following steps:
a) applying to the hair a shampoo composition comprising a detersive surfactant, wherein the shampoo composition is substantially free of silicone conditioning agents and cationic conditioning polymers;
b) rinsing the shampoo composition from the hair;
c) applying to the hair a conditioning composition comprising a silicone resin and an aminosilicone, wherein the aminosilicone has non-quaternized and/or quaternary amine functional groups.

4. The method of claims 1-3, wherein the step (c) is: applying to the hair a conditioning composition comprising a silicone resin and an aminosilicone, wherein the aminosilicone has non-quaternized and/or quaternary amine functional groups, and rinsing the conditioning composition from the hair.

5. The method of claims 1-4, further comprising a step:
d) applying an additional conditioning composition to the hair, and rinsing the additional conditioning composition from the hair.

6. The method of claims 1-4, further comprising a step:
e) applying an additional leave-on conditioning composition to the hair.

7. The method of claims 1-3, wherein no composition is applied between the step (b) “rinsing the shampoo composition from the hair” and the step (c) “applying to the hair the conditioning composition”.

8. The method of claims 1-3, wherein the aminosilicone has non-quaternized amine functional groups.

9. The method of claims 1-3, wherein the aminosilicone has the amine functional groups: (i) as pendant groups attaching to a polysiloxane backbone; or (ii) at terminal ends of a polysiloxane backbone

10. The method of claims 1-3, wherein the conditioning composition further comprises: a cationic surfactant; a high melting point fatty compound; and an aqueous carrier.
